(19) United States
(12) Patent Application Publication
(10) Pub. No.: US 2010/0081131 A1
(43) Pub. Date: Apr. 1, 2010

(54) IDENTIFICATION OF MICROBES USING OLIGONUCLEOTIDE BASED IN SITU HYBRIDIZATION

(76) Inventors: Robert A. Ach, San Francisco, CA (US); N. Alice Yamada, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 12/243,585

(22) Filed: Oct. 1, 2008

(51) Int. Cl. C12Q 1/68

(52) U.S. Cl. 435/6

(57) ABSTRACT

A method of sample analysis is provided. In certain embodiments, the method may comprise: a) contacting a sample comprising a microbe with a set of at least two labeled oligonucleotides under in situ hybridization conditions to produce a contacted sample, where the labeled oligonucleotides i. hybridize to different RNA molecules of the microbe at sites that are unique to the microbe, ii. provide a predetermined optically detectable signature that identifies the microbe when the labeled oligonucleotides are hybridized to the different RNA molecules of the microbe, and iii. do not hybridize to ribosomal RNA of the microbe; b) reading the contacted sample to detect hybridization of the labeled oligonucleotides; and c) determining the identity of the microbe on the basis of the predetermined optically detectable signal, where the predetermined optically detectable signal indicates the identity of the microbe in the sample.

### BACKGROUND

The rapid identification of microbes is of great importance in clinical diagnosis, public health, veterinary health, biodefense, environmental science, and agriculture. Microbes can be identified and classified on the basis of their shape, growth characteristics, nutrient requirements, metabolic activity, presence of certain genes, expression of certain genes, etc. The process for separation and identification of microbes is largely dominated by 19th century procedures of growing and isolating pure cultures. This is a slow and tedious process that works only for a small fraction of microbes. There are many microbes that still cannot be isolated and identified in this manner. Furthermore, such a process does not allow for the rapid differentiation between various microbes in a complex mixture nor quantification and evaluation of the microbes.

There is a constant demand in the art for methods to identify microbes. Certain aspects of this disclosure relate to such methods.

### SUMMARY

A method of sample analysis is provided. In certain embodiments, the method may comprise: a) contacting a sample comprising a microbe with a set of at least two labeled oligonucleotides under in situ hybridization conditions to produce a contacted sample, where the labeled oligonucleotides i. hybridize to different RNA molecules of the microbe at sites that are unique to the microbe, ii. provide a predetermined optically detectable signature that identifies the microbe when the labeled oligonucleotides are hybridized to the different RNA molecules of the microbe, and iii. do not hybridize to ribosomal RNA of the microbe; b) reading the contacted sample to detect hybridization of the labeled oligonucleotides; and c) determining the identity of the microbe on the basis of the predetermined optically detectable signal, where the predetermined optically detectable signal indicates the identity of the microbe in the sample.

### BRIEF DESCRIPTION OF THE DRAWINGS

FIG. 1 shows a schematic of an embodiment described herein.

FIG. 2 shows a schematic of certain features of some embodiments of a method described herein.

FIG. 3 shows a schematic of an embodiment of the subject method described herein.

### DEFINITIONS

The term “sample” as used herein relates to a material or mixture of materials, typically, although not necessarily, in liquid form, containing one or more analytes of interest.

The term “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” refers to a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively).

The term “oligonucleotide” as used herein denotes a single stranded multimer of nucleotide of from about 2 to about 200 nucleotides. Oligonucleotides may be synthetic or may be made enzymatically, and, in some embodiments, are under 10 to 50 nucleotides in length. Oligonucleotides may contain ribonucleotide monomers (i.e., may be oligoribonucleotides) or deoxyribonucleotide monomers. Oligonucleotides may be 10 to 20, 11 to 30, 31 to 40, 41 to 50, 51-60, 61 to 70, 71 to 80, 80 to 100, 100 to 150, 150 to 200 or 200-250 nucleotides in length, for example 150 nucleotides.

An “array,” includes any two-dimensional or substantially two-dimensional (as well as a three-dimensional) arrangement of addressable regions, e.g., spatially addressable regions or optically addressable regions, bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a surface of the substrate. Depending upon the use, any or all of the arrays may be the same or different from one another and each may contain multiple spots or features. An array may contain one or more, including more than two, more than ten, more than one hundred, more than one thousand, more ten thousand features, more than one hundred thousand features, up to one million features, or more, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 mm and less than 1.5 mm, such as more than about 0.8 mm and less than about 1.2 mm.

Arrays can be fabricated using drop deposition from pulse-jets of either precursor units (such as nucleotide or amino acid monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

An array is “addressable” when it has multiple regions of different moieties (e.g., different oligonucleotide sequences) such that a region (i.e., a “feature” or “spot” of the array) at a particular predetermined location (i.e., an “address”) on the array contains a particular sequence. Array features are typically, but need not be, separated by intervening spaces.

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Determining the presence of” includes determining the amount of something present, as well as determining whether it is present or absent. “Determining the identity” includes assigning something a descriptor that identifies it, e.g., determining the identity of a microbe refers to assigning it a descriptor that indicates its common name, scientific name, code, family, genus, species, strain, or genotype.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

The term “microbe”, as used herein, refers to a microorganism. The term includes bacteria, fungi, archaea, and protists. The term “microbe” includes pathogenic bacteria, causing diseases such as plague, tuberculosis and anthrax; protozoa, causing diseases such as malaria, sleeping sickness and toxoplasmosis; and also fungi causing diseases such as ringworm, candidiasis or histoplasmosis, for example.

The term “in situ” refers to “inside a cell”. For example, the RNA being detected by in situ hybridization is present inside a cell. The cell may be permeabilized or fixed, for example.

The term “hybridization” refers to the specific binding of a nucleic acid to a complementary nucleic acid via Watson-Crick base pairing. Accordingly, the term “in situ hybridization” refers to specific binding of a nucleic acid to a complementary nucleic acid inside a cell.

The terms “hybridizing” and “binding”, with respect to nucleic acids, are used interchangeably.

The term “contacting” means to bring or put together. As such, a first item is contacted with a second item when the two items are brought or put together, e.g., by touching them to each other or combining them in the same solution.

The term “in situ hybridization conditions” as used herein refers to conditions that allow hybridization of a nucleic acid to a complementary nucleic acid, e.g., a sequence of nucleotides in a RNA molecule and a complementary oligonucleotide, in a cell. Suitable in situ hybridization conditions may include both hybridization conditions and optional wash conditions, which conditions include temperature, concentration of denaturing reagents, salts, incubation time, etc. Such conditions are known in the art.

The terms “ribonucleic acid” and “RNA” as used herein refers to a polymer composed of ribonucleotides.

The phrase “different RNA molecules” as used herein refers to RNA molecules that have different nucleotide sequences, e.g., different RNA molecules are transcribed from different genes.

The term “sites”, as used in the context of a site in a nucleic acid molecule, refers to a contiguous sequence of nucleotides in the nucleic acid molecule.

The phrase “labeled oligonucleotide” refers to an oligonucleotide that contains a detectable moiety. The detectable moiety may produce a signal directly or indirectly. One example of a detectable moiety that produces a signal directly is a fluorescent molecule. Detectable moieties that produce a signal indirectly include moieties that produce a signal upon exposure to detection reagents such as substrates or antibodies, etc. A detectable moiety that produces a signal directly can optionally be detected by indirect means such as by using a labeled antibody that binds to the moiety. In certain cases, a signal may be of a particular wavelength which is detectable by a photodetector, e.g., a light microscope, a spectrophotometer, a fluorescent microscope, a fluorescent sample reader, or a florescence activated cell sorter, etc.

The term “unique” refers to a characteristic that is only found in members of one type of a class, species, etc. For example, “a binding site unique to a microbe” or a grammatical equivalent thereof, refers to a contiguous sequence of nucleotides that is found only in microbes that belong to the same genus, same species, or same strain. Thus, a unique sequence allows the identification of a microbe to a particular genus, species, or strain.

The term “predetermined” refers to something that is known before use. The phrase “predetermined signature” refers to a signature that it is known before use.

The phrase “optically detectable signature” refers to a light signal that can be detected by a photodetector, e.g., a light microscope, a spectrophotometer, a fluorescent microscope, a fluorescent sample reader, or a florescence activated cell sorter, etc. “Optically detectable signature” may be made up of one or more signals, where the signal(s) is produced by a label(s). An optically detectable signature may be made up of: a single signal, a combination of two or more signals, ratio of magnitude of signals, etc. The signal may be visible light of a particular wavelength. An optically detectable signature may be a signal from a fluorescent label(s). For example, the “optically detectable signature” for Cy5 is a visible light at the wavelength of 670 nm.

The phrase “plurality of sets of labeled oligonucleotides” means two or more sets of oligonucleotides where each set comprises at least two labeled oligonucleotides.

The phrase “different microbes” is used interchangeably with “different types of microbes”. These phrases refer to microbes that are distinct from each other because they belong to a different genus, or to a different species or to a different strain. Two microbes that belong to different genus are considered to be different, microbes that belong to the same genus but to different strains are considered to be different, microbes that belong to the same genus and species but to different strains are also considered to be different.

The phrase “associated with” refers to the situation where a characteristic of a first thing is imparted to a second thing such that the second thing then has that characteristic. For example, a signal associated with a microbe refers to a signal that comes from the microbe by virtue of labeled oligonucleotides being hybridized to the RNA of the microbe. Similarly, an optically detectable signature associated with a microbe refers to the signature which the microbe has by virtue of labeled oligonucleotides being hybridized to the RNA of the microbe.

The term “matching” refers to the process of comparing one thing to another to find a match. For example, the optically detectable signal associated with a microbe is compared to that associated with a list of known microbes.

The terms “plurality”, “set” or “population” are used interchangeably to mean at least 2, at least 10, at least 100, at least 500, at least 1000, at least 10,000, at least 100,000, up to at least 1,000,000, or 10,000,000 or more.

The phrase “distinguishable labels” or any grammatical equivalent thereof refers to labels can be independently detected and measured, even when the labels are mixed. In other words, the amounts of label present (e.g., the amount of fluorescence) for each of the labels are separately determinable, even when the labels are co-located (e.g., in the same tube or in the same duplex molecule or in the same cell). Suitable distinguishable fluorescent label pairs include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), and POPRO3 and TOPRO3 (Molecular Probes, Eugene, Oreg.). Further suitable distinguishable detectable labels may be found in Kricka et al. (Ann Clin Biochem. 39:114-29, 2002).

The term “probes” as used herein refers to labeled oligonucleotides that hybridize to complementary nucleic acid sequences under in situ hybridization conditions. Thus rRNA probes refer to labeled oligonucleotides that hybridize to complementary rRNA sequences.

The phrase “high copy number RNA” refers to an RNA that is present in multiple copies in a cell such that it accounts for a significant portion of the total RNA expressed in the cell. A high copy number RNA may account for at least 5%, at least 10%, at least 20% or at least 50% of the total RNA population of a cell. The phrase “low copy number RNA” is present in very few copies in a cell such that it does not account for a significant portion of the total RNA present in the cell. A low copy number RNA may account for less than 5%, less than 2%, less than 1%, less than 0.1% or less than 0.05%, or lesser of the total RNA population of a cell. rRNA is an example of a high copy number RNA while many messenger RNAs (mRNA) are low copy number RNAs.

### DESCRIPTION OF EXEMPLARY EMBODIMENTS

A method of sample analysis is provided. In certain embodiments, the method may comprise: a) contacting a sample comprising a microbe with a set of at least two labeled oligonucleotides under in situ hybridization conditions to produce a contacted sample, where the labeled oligonucleotides i. hybridize to different RNA molecules of the microbe at sites that are unique to the microbe, ii. provide a predetermined optically detectable signature that identifies the microbe when the labeled oligonucleotides are hybridized to the different RNA molecules of the microbe, and iii. do not hybridize to ribosomal RNA of the microbe; b) reading the contacted sample to detect hybridization of the labeled oligonucleotides; and c) determining the identity of the microbe on the basis of the predetermined optically detectable signal, where the predetermined optically detectable signal indicates the identity of the microbe in the sample.

Before the present subject invention is described further, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a microbe” includes a plurality of microbes and reference to “RNA sequence” includes reference to one or more RNA sequences and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Method for Sample Analysis

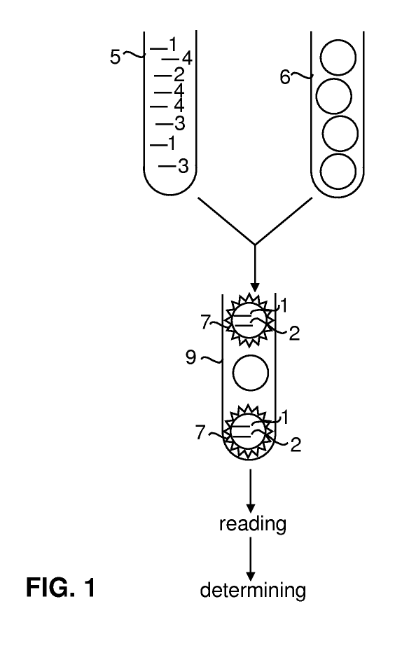

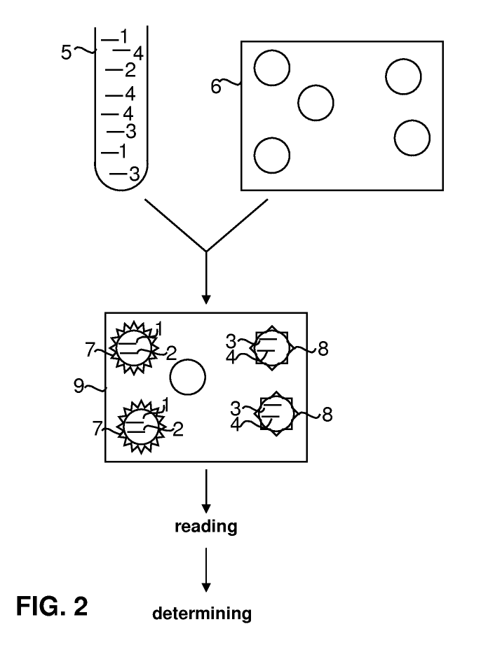

Certain features of the subject method are illustrated in FIG. 1. With reference to FIG. 1, the method generally includes contacting sample 6 with a plurality of sets of labeled oligonucleotides 5, under in situ hybridization conditions, to produce a contacted sample 9. The sample may contain one type of microbe or different types of microbes. The plurality of sets of labeled oligonucleotides may be present separately or may be mixed together. Each set contains at least two labeled oligonucleotides. In FIG. 1, labeled oligonucleotides 1 and 2 are from a set; labeled oligonucleotides 3 and 4 are from another set. Labeled oligonucleotides 1 and 2 (and any of the other oligonucleotides used in identifying microbes by the subject method) do not hybridize to ribosomal RNA of microbes. Labeled oligonucleotides 1 and 2 hybridize to different RNA molecules of a microbe at sites that are unique to the microbe. Labeled oligonucleotides 1 and 2 provide a predetermined optically detectable signature 7 that identifies the microbe when the labeled oligonucleotides 1 and 2 are hybridized to the different RNA molecules of the microbe. This microbe is also referred to as the target microbe for this set of labeled oligonucleotides. In FIG. 1, the target microbe for labeled oligonucleotides 3 and 4 is not present. The sample might contain microbes that are suspended in a solution (FIG. 1) or the microbes may be immobilized on a substrate (FIG. 2). In certain embodiments, the sample may be a tissue section. In other embodiments, the sample may be a sewage sample that may either be a suspension or deposited on a substrate. Following hybridization, the contacted sample 9 is read to detect hybridization of the labeled oligonucleotides. The reading step detects the predetermined optically detectable signature associated with the microbe in the contacted sample. The reading step is followed by determining the identity of the microbe on the basis of the predetermined optically detectable signature, where the signature indicates the identity of the microbe. The microbe may be identified by matching the predetermined optically detectable signature associated with the microbe in the contacted sample to optically detectable signature associated with known microbes.

In embodiments where the labeled oligonucleotides in a set are labeled with the same label, the hybridization of the oligonucleotides of this set to the target microbe produces a single signal which is read to provide the predetermined optically detectable signature to the target microbe. In this embodiment, the predetermined optically detectable signature is made of one signal. In this embodiment, the labeled oligonucleotides of a set have the same label which is distinguishable from the label of the labeled oligonucleotides of another set. Thus, when a plurality of sets are used, where each set hybridizes to RNA molecules of different microbes, the hybridization of the labeled oligonucleotides of a set to the target microbe produces a single signal which provides the predetermined optically detectable signature. This signature is distinguishable from that associated with a different microbe.

In certain embodiments, a set of oligonucleotides may include a first and a second population of labeled oligonucleotides. The first population is labeled with a first label that produces a first signal and the second population of labeled oligonucleotides is labeled with a second label that produces a second signal that is distinguishable from the first signal. The hybridization of this set to a target microbe provides an optically detectable signature to the microbe where the signature is the combination of the first and the second signal. Similarly, another set of oligonucleotides may include a first and a second population of labeled oligonucleotides. The first population in this set is labeled with a first label that produces a first signal and the second population of labeled oligonucleotides is labeled with a third label that produces a third signal that is distinguishable from the first and the second signals. The hybridization of this set to the target microbe provides an optically detectable signature to the microbe where the signature is the combination of the first and the third signals. Various combinations of labels may be used in conjunction with dividing a set of oligonucleotides into two or more populations such that the microbe that the set binds to has a unique combination of signals and hence a unique optically detectable signature. The label(s) of the oligonucleotides, the signal from the labeled oligonucleotides, the binding of the oligonucleotides to different RNA molecules of a microbe and the signals associated with the microbe are all predetermined leading to a predetermined optically detectable signature for each type of microbe.

In certain embodiments, the reading step may include determining the ratio of the magnitudes of two or more signals associated with a microbe to which labeled oligonucleotides are hybridized. In certain embodiments, a set of labeled oligonucleotides may comprise a first population of labeled oligonucleotides and a second population of labeled oligonucleotides. The first population is labeled with a first label that produces a first signal and the second population of labeled oligonucleotides is labeled with a second label that produces a second signal that is distinguishable from the first signal. In this embodiment, it may be the ratio of the magnitudes of the two signals which provides the optically detectable signature. The ratio of the magnitudes of the signals is determined by the amounts of labeled oligonucleotides with a particular label that are hybridized to the target microbe. For example, if the first and second populations have equal amounts of labeled oligonucleotides and the oligonucleotides in the first population are labeled with a first label and those in second population are labeled with a second label, when these labeled oligonucleotides hybridize to their target microbe, the microbe has a predetermined optically detectable signature which is the ratio of the magnitudes of the signals which in this example would be 1:1. In yet another embodiment, the number of oligonucleotides in the first and second populations in a set might be chosen such that each set has a first population and a second population where the amount of oligonucleotides in the populations is different from the amount of oligonucleotides in the first and second populations in another set. Accordingly the magnitude of signals from the first label and second label is different for each set, resulting in sets that each provide an optically detectable signature to the microbe they target.

As an example, a certain strain of E. coli may be known to be present in a sample and that strain is suspected to be K12-MG1655. In such cases, a set of labeled oligonucleotides that binds to RNA molecules at sites unique to that strain may be used. In this scenario, all of the labeled oligonucleotides in the set might have the same label and provide a predetermined optically detectable signature to that strain of E. coli, where the signature is made up of the single signal produced by the label. Alternatively, the set might also divided be into a first population and a second population. The first population labeled with a first label that produces a first signal and the second population of labeled oligonucleotides labeled with a second label that produces a second signal that is distinguishable from the first signal. The hybridization of this set to the target E. coli strain provides an optically detectable signature to the microbe where the signature is the combination of the first and the second signals.

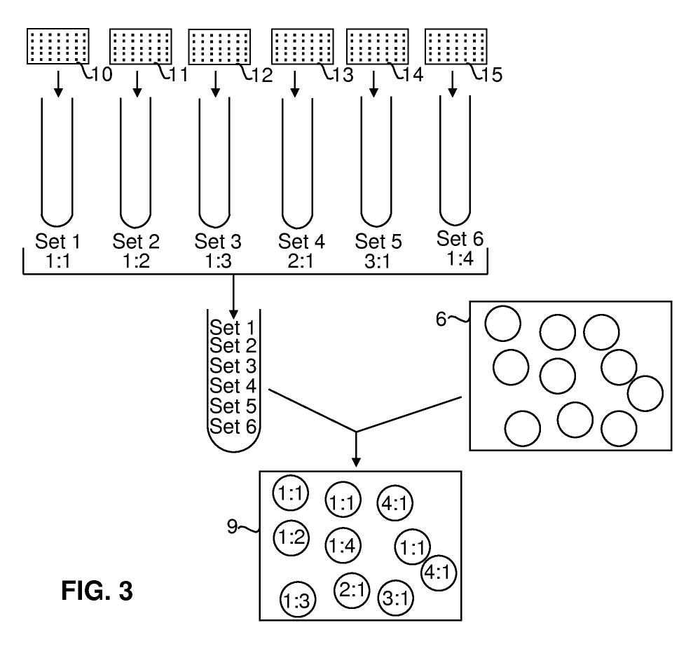

In certain embodiments a plurality of sets of labeled oligonucleotides may be used to identify a microbe, where it is not known what type of microbe is present in a sample. Each set of labeled oligonucleotides in a plurality of sets binds to RNA molecules of different microbes and provides a predetermined optically detectable signature that identifies the different microbes. In this embodiment the predetermined optically detectable signature associated with one microbe is distinguishable from that associated with another microbe. For example, if it is known that different strains of E. coli are present in a sample but it is not known what those strains may be, then a plurality of sets of labeled oligonucleotides that identify different strains of E. coli may be used. In this example, each strain has a predetermined optically detectable signature that is distinguishable from that of another strain. For example, if there are five sets of labeled oligonucleotides where each set binds to a different strain of E. coli, each set is labeled such that when the sets hybridize to target strain, a different predetermined optically detectable signature is associated with a different strain. In one embodiment, the labeled oligonucleotides of a set have a single label which is distinguishable from the label of the labeled oligonucleotides of another set. Thus in this example, the five labels may be used to distinguishably label the oligonucleotides in the five sets. Alternatively, the oligonucleotides in each set may be divided into two populations. The first population and second population in set 1 can be labeled with a first and second label, respectively. In set 2, the first population and second population can be labeled with the first and a third label, respectively. In set 3, the first population and second population can be labeled with the first and a fourth label, respectively. In set 4, the first population and second population can be labeled with the second and the third label, respectively. In set 5, the first population and second population can be labeled with the second and the fourth label, respectively. Each of these labels produce signals that are distinguishable from each other. When these sets hybridize to their target microbe, each microbe has a different signature comprised of the different combination of signals. Alternatively, the oligonucleotides in each set may be divided into two populations such that each set has a first population and a second population where the number of oligonucleotides in the populations is different from the number of oligonucleotides in the first and second populations in another set. Accordingly the magnitude of signals from the first label and second label is different for each set, resulting in sets that each provide an optically detectable signature to the microbe they target. For example, in set 1, the amount of oligonucleotides in the first population is equal to the amount of oligonucleotides in the second population. In set 2, the amount of oligonucleotides in the first population is half the amount of oligonucleotides in the second population. In set 3, the amount of oligonucleotides in the first population is double the amount of oligonucleotides in the second population. In set 4, the amount of oligonucleotides in the first population is triple the amount of oligonucleotides in the second population. In set 5, the amount of oligonucleotides in the first population is a third of the amount of oligonucleotides in the second population. When these sets hybridize to their target microbe, each microbe has a different signature comprised of the ratio of the magnitudes of the first and the second signals. In this example, hybridization of sets 1-5 would produce signatures that have the ratio of signal from first label to signal from second label as 1:1, 1:2, 2:1, 3:1 and 1:3, respectively. A similar embodiment is depicted in FIG. 3.

In certain cases, the sets of oligonucleotides used in the subject method may be pre-tested against microbes of known identities. Thus, the labels for the oligonucleotides, the differently labeled populations in each set, the target sequences for each set, the optically detectable signature provided by each set to microbes of known identity is predetermined. In other embodiments, the optically detectable signature may be determined in silico, e.g., by comparing each set of oligonucleotides to gene expression data for a microbe stored in the memory of a computer. The optically detectable signature associated with a known microbe when a certain set(s) of labeled oligonucleotides is used is stored as a list or a look up table. This list or look up table or any similar data storage format is used for matching the predetermined optically detectable signature associated with the microbe to be identified to that of microbe of known identity.

Oligonucleotides used in the subject method may be about 10 to 20, 11 to 30, 31 to 40, 41 to 50, 51-60, 61 to 70, 71 to 80, 80 to 100, 100 to 150, 150 to 200 nucleotides in length, for example 150 nucleotides. In certain embodiments the oligonucleotides are under 12-50 nucleotides in length. In certain other embodiments, the oligonucleotides are under 30-100 nucleotides in length. In yet other embodiments, the oligonucleotides are under 30-200 nucleotides in length.

Oligonucleotides used in the subject method may be designed by utilizing the genome sequence information as well as expressed gene sequence information available at several public and private databases, for example. For example, genomic sequence information is available via Microbe Genome Sequencing Project, Department of Energy, U.S.A. and from NCBI. Expressed gene sequence information is available at GenBank. Additionally, expressed gene sequences can be derived from gene expression profiling of microbes of interest. Microarrays representing the genome of a variety of microbes as well as custom microarrays for microbes of interest are available from numerous vendors.

Oligonucleotides used in the subject method hybridize to different RNA molecules of a microbe at sites that are unique to the microbe. These oligonucleotides do not hybridize to ribosomal RNA. In certain cases, these oligonucleotides do not hybridize to a high copy number RNA. A RNA is deemed to be a high copy number RNA if the RNA accounts for at least 5%, at least 10%, at least 20% or at least 50% of the total RNA population of a microbe. These oligonucleotides hybridize to low copy number RNA. A RNA is deemed to be a low copy number RNA if it accounts for less than about 5%, less than 2%, less than 1%, less than 0.1% or less than 0.05%, or lesser of the total RNA population of a cell.

The oligonucleotides can optionally be amplified prior to hybridization. Suitable amplification methods include, but are not limited to polymerase chain reaction (PCR) (Innis, et al., PCR Protocols: A guide to Methods and Application, Academic Press Inc., San Diego, (1990)), ligase chain reaction (LCR) (see Wu and Wallace,Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87: 1874 (1990)).

The oligonucleotides used in the subject method may be labeled. The labels may be incorporated by any of a number of means well known to those of skill in the art. The label may be simultaneously incorporated during the amplification step. Thus, for example, polymerase chain reaction (PCR) with labeled primers or labeled nucleotides will provide a labeled amplification product. In certain embodiment, a label may be added directly to the oligonucleotides or to the amplification product after the amplification is completed. Means of attaching labels to nucleic acids are well known to those of skill in the art and include, for example nick translation or end-labeling, by kinasing of the nucleic acid and subsequent attachment of a nucleic acid linker joining the oligonucleotides to a label. Standard methods may be used for labeling the oligonucleotide, for example, as set out in Maniatis et al, Molecular Cloning, A Laboratory Manual, Cold Spring Harbor Publication (1982).

Detectable labels suitable for use in the present method, compositions and kits include any label detectable by spectroscopic, photochemical, biochemical, immunochemical, electrical, optical or chemical means. Useful labels include biotin for staining with labeled streptavidin conjugate, magnetic beads (e.g., Dynabeads™), fluorescent dyes (e.g., fluorescein, texas red, rhodamine, green fluorescent protein, cyanins and the like), radiolabels (e.g., 3H, 35S, 14C, or 32P, enzymes (e.g., horseradish peroxidase, alkaline phosphatase and others commonly used in ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241, which are herein incorporated by reference.

Oligonucleotides useful in the subject methods may be comprised in sets. In certain embodiments, a set of oligonucleotides may contain at least 10-100 oligonucleotides. In certain embodiments, a set of oligonucleotides may contain at least 100-1000 oligonucleotides. In certain embodiments, a set of oligonucleotides may contain at least 1000-10,000 oligonucleotides, or more. A set of oligonucleotides may contain oligonucleotides that bind to different RNA molecules of a single type of microbe. In certain embodiments, the oligonucleotides of a set may be designed to overlap with each other. In some cases, the amount of overlap may be dependent upon the length of the oligonucleotides. For example, for oligonucleotides that are about 20 nucleotides long, the overlap may be at least one nucleotide from one oligonucleotide to the next. In certain embodiments the overlap may be two or more nucleotides. For oligonucleotides that are about 100 nucleotides long, the overlap may be at least 20 nucleotides from one oligonucleotide to the next. In certain embodiments the overlap may be 50 or more nucleotides. In certain embodiments the overlap may be up to 90 nucleotides. In other embodiments, the oligonucleotides of a set may be designed to be end-to-end tiled. A plurality of such sets will provide oligonucleotides that target different types of microbes. Thus, oligonucleotides of a first set might bind to different RNA molecules at sites that are unique to of a first microbe; oligonucleotides of a second set might bind to different RNA molecules of a second microbe, and so on. A plurality of sets may be at least 2, at least 10, at least 100, at least 500, at least 1000, at least 10,000, at least 100,000, or up to 1000,000 or more sets. In certain embodiments, a plurality of sets may be at least 10-50 sets. In certain embodiments, a plurality of sets may be at least 51-100 sets. In certain embodiments, a plurality of sets may be at least 101-1000 sets. In certain embodiments, a plurality of sets might be mixed together.

The oligonucleotides of a set may be present in a solution or attached to an array. In embodiments where the oligonucleotides of a set are attached to an array, the oligonucleotides are cleaved off before use in subject method. The oligonucleotides in a set might be labeled or unlabeled. In cases, where oligonucleotides are unlabeled, the oligonucleotides may be labeled before use in the subject method. When a plurality of sets of oligonucleotides is used, each of the sets may be in a separate container (tube or vessel or well) or the sets might be mixed together in a single container. When a plurality of sets of oligonucleotides is used, each of the set may be attached to a separate array. Such an embodiment is depicted in FIG. 3. Each of the arrays may be present as a single array on a chip. Alternatively, multiple copies of the same array or multiple different arrays might be present on a single chip. In certain cases, sets of oligonucleotides may be attached to an array with a cleavable linker that is cleaved to release a mixture of oligonucleotides.

In general, methods for oligonucleotides synthesis and purification, as well as methods for the preparation of oligonucleotide arrays are well known in the art (see, e.g., Harrington et al., Curr. Opin. Microbiol. 2000, 3, 285-91 and Lipshutz et al., Nat. Genet. 1999, 21:20-24) and need not be described in any great detail. Oligonucleotides can be synthesized, for example, on a Perkin Elmer-Applied Biosystems 381A DNA synthesizer using standard automated phosphoramidite chemistry. Oligonucleotides can be fabricated using any means, including drop deposition from pulse jets or from fluid-filled tips, etc., or using photolithographic means. Oligonucleotide precursor units (such as nucleotide monomers), in the case of in situ fabrication, can be deposited. Such methods are described in detail in, for example, U.S. Pat. Nos. 6,242,266, 6,232,072, 6,180,351, 6,171,797, 6,323,043, and U.S. Patent Application US20040086880 A1, etc., the disclosures of which are herein incorporated by reference. In certain cases, oligonucleotides can be attached to an array via a cleavable linker. Such arrays are described in U.S. Pat. No. 7,291,471, herein incorporated by reference.

In embodiments using a set of labeled oligonucleotides, all of the labeled oligonucleotides in a set may comprise the same label. In this embodiment, the labeled oligonucleotides of a set have the same label which is distinguishable from the label of the labeled oligonucleotides of another set.

In embodiments using a set of labeled oligonucleotides, the set may comprise a first population of labeled oligonucleotides and a second population of labeled oligonucleotides, where the first population is labeled with a first label that produces a first signal and the second population is labeled with a label that produces a second signal that is distinguishable from the first signal. In embodiments where a plurality of sets of labeled oligonucleotides is used, oligonucleotides in each of the sets are labeled with a labeling strategy that utilizes combinations of the variety of distinguishable labels available for labeling oligonucleotides. Thus, sets of labeled oligonucleotides may include a first and a second population of labeled oligonucleotides. The first population in a set could be labeled with a first label that produces a first signal and the second population of labeled oligonucleotides could be labeled with a second label that produces a second signal that is distinguishable from the first signal. The first population in another set could be labeled with a first label that produces a first signal and the second population of labeled oligonucleotides could be labeled with a third label that produces a third signal that is distinguishable from the first and the second signals. In yet another set of labeled oligonucleotides, first population may be labeled with a first label that produces a first signal and the second population of labeled oligonucleotides may be labeled with a fourth label that produces a fourth signal that is distinguishable from the first, second and third signals, and so on.

In certain embodiments, a set of labeled oligonucleotides might comprise a first population of labeled oligonucleotides and a second population of labeled oligonucleotides. The first population is labeled with a first label that produces a first signal and the second population of labeled oligonucleotides is labeled with a second label that produces a second signal that is distinguishable from the first label. The magnitude of a signal is dependent on the amount of labeled oligonucleotides in a population. Thus, when the first and second populations have the same amount of labeled oligonucleotides, the magnitude of the first signal will be the same as the second signal and the ratio of the magnitudes will be 1:1. Thus, the ratio of the magnitudes of the signal can be changed by changing the amount of labeled oligonucleotides in a population. FIG. 3 shows an embodiment of a method using different sets of labeled oligonucleotides, where each set provides a different predetermined optically detectable signature to the microbe that the set binds to. The different predetermined optically detectable signatures are different ratios of the magnitude of a first signal to the magnitude of a second signal. This method may employ two distinguishable labels when a set is divided into two populations. Alternatively, in some embodiments, more than two labels might be employed when a set of oligonucleotides is divided into more than two populations. With reference to FIG. 3, each set of oligonucleotides is present on a single array, and each array comprises a single set of oligonucleotides. In other embodiments, an array may comprise multiple sets of oligonucleotides, where the oligonucleotides of a single set can be amplified using PCR methods. For example, the oligonucleotides of each set may have PCR primer binding sites that differ to oligonucleotides of other sets. A first PCR primer pair and a second PCR primer pair are used to amplify a first population and a second population of oligonucleotides, respectively. The two populations are separated and distinguishably labeled. The labeled population of oligonucleotides are optionally mixed together and contacted to the sample or sequentially contacted to the sample.

In certain embodiments, the two or more populations of oligonucleotides of a set may be present on different arrays. These populations of oligonucleotides of a set may then be cleaved off and distinguishably labeled. Alternatively, these populations of oligonucleotides of a set may be amplified and labeled sequentially or simultaneously. The labeled population of oligonucleotides are optionally mixed together and contacted to the sample or sequentially contacted to the sample.

Optically detectable signature refers to a light signal that can be detected by a photodetector. Optically detectable signature may be made up of one or more signals, where the signal is produced by a label. Optically detectable signature includes: a single signal, a combination of two or more signals, ratio of magnitude of signals, etc. The signal may be visible light of a particular wavelength. An optically detectable signature may be provided by a fluorescent signal(s).

Optically detectable signatures used to identify a microbe are predetermined, i.e., a certain optically detectable signature would be present if a certain type of microbe is present in the sample. Optically detectable signatures are predetermined by hybridizing sets of labeled oligonucleotides to known microbes, or else are predetermined using in silico calculations. In silico calculations may be used to provide predetermined optically detectable signatures for microbes that are hard to culture and are consequently available in limited quantities.

In certain embodiments, all of the labeled oligonucleotides in a set may comprise the same label. When a plurality of sets is used, the label of labeled oligonucleotides of a set is distinguishable from the label of the labeled oligonucleotides of another set. For example, when a plurality of sets is used where each set binds to a different microbe, oligonucleotides of a first set might be labeled with Cy5, oligonucleotides of a second set might be labeled with Cy3, oligonucleotides of a third set might be labeled with Alexa Fluor 350, oligonucleotides of a fourth set might be labeled with Alexa Fluor 488, and so on. For example, the optically detectable signal for Cy5 is a visible light at the wavelength of 670 nm. Thus when all labeled oligonucleotides of a set are labeled with Cy5 and hybridized to RNA molecules at sites that are unique to microbe A, then any microbe associated with an optically detectable signature that is a visible light at the wavelength of 670 nm will be identified as microbe A. In this manner, the optically detectable signature is predetermined.

In certain embodiments, a set of oligonucleotides may include a first and a second population of labeled oligonucleotides. The first population is labeled with a first label that produces a first signal and the second population of labeled oligonucleotides is labeled with a second label that produces a second signal that is distinguishable from the first signal. The hybridization of this set to a target microbe provides an optically detectable signature to the microbe where the signature is the combination of the first and the second signal. Thus, for example, set 1, whose target microbe is microbe A, is divided into two populations, where the first population is labeled with Cy3 and the second population is labeled with Cy5. When this set is hybridized to microbe A, microbe A will have an optically detectable signature that is a combination of signals of wavelength 570 nm (from Cy3) and wavelength 670 nm (from Cy5). Thus, any microbe in a contacted sample having the predetermined optically detectable signature that is a combination of signals of wavelength 570 nm (from Cy3) and wavelength 670 nm (from Cy5) would be identified as microbe A. Similarly, for example, if in a set, the first population is labeled with Cy3 and the second population is labeled with Alexa Fluor 346, the microbe to which this set binds to will have an optically detectable signature that is a combination of signals of wavelength 570 nm (from Cy3) and wavelength 442 nm (from Alexa Fluor 346). Thus, the sets are labeled in a manner such that the combination of signals is unique to a particular type of microbe, providing a predetermined optically detectable signature to that type of microbe.

In certain other embodiments, a set of oligonucleotides may include a first and a second population of labeled oligonucleotides. The first population is labeled with a first label that produces a first signal and the second population of labeled oligonucleotides is labeled with a second label that produces a second signal that is distinguishable from the first signal. The hybridization of this set to a target microbe provides an optically detectable signature to the microbe where the signature is the ratio of the magnitude of the first signal to the magnitude of the second signal. Thus when the magnitudes of the two signals is same the ratio is 1:1, which would be the optically detectable signal for the microbe to which the set is bound. Similarly, another set might be labeled such that when the set is bound to the target microbe, ratio of the magnitudes of the two signals would be 1:2, then the optically detectable signal for the microbe to which the set is bound would be 1:2, and so on. The sets of labeled oligonucleotides would be tested on known microbes to determine the optically detectable signal for the microbes. This testing would provide the list or database or look up table for the predetermined optically detectable signals.

Methods for collecting and storing biological and non-biological samples are generally known to those of skill in the art. For example, the Association of Analytical Communities International (AOAC International) publishes and validates sampling techniques for testing foods and agricultural products for microbial contamination. See also WO 98/32020 and U.S. Pat. No. 5,624,810, which set forth methods and devices for collecting and concentrating microbes from the air, a liquid, or a surface. WO 98/32020 also provides methods for removing somatic cells, or animal body cells present at varying levels in certain samples.

In certain cases, a separation and/or concentration step may be necessary to separate microbial organisms from other components of a sample or to concentrate the microbes to an amount sufficient for rapid detection. For example, a sample suspected of containing a microbial organism may require a selective enrichment of the organism (e.g., by culturing in appropriate media, e.g., for 6-96 hours or longer). Alternatively, appropriate filters and/or immunomagnetic separations can concentrate a microbial pathogen without the need for an extended growth stage. For example, antibodies specific for a microbial antigen can be attached to magnetic beads and/or particles. Multiplexed separations, in which two or more concentration processes are employed may also be used, e.g., centrifugation, membrane filtration, electrophoresis, ion-exchange, affinity chromatography, and immunomagnetic separations.

Certain air or water samples may need to be concentrated. For example, certain air sampling methods require the passage of a prescribed volume of air over a filter to trap any microbial organisms, followed by isolation of the microbe(s) into a buffer or liquid culture. Alternatively, the focused air is passed over a plate (e.g., agar) medium for growth of any microbial organisms.

Methods for sampling a tissue with a swab are known to those of skill in the art. Generally, a swab is hydrated (e.g., with an appropriate buffer, such as Cary-Blair medium, Stuart's medium, PBS, buffered glycerol saline, or water) and used to sample an appropriate surface (e.g., a tissue) for a microbial organism. Any microbe present is then recovered from the swab, such as by centrifugation of the hydrating fluid away from the swab, removal of supernatant, and resuspension of centrifugate in an appropriate buffer, or by washing of the swab with additional diluent or buffer. The recovered sample then may be analyzed according to the methods described herein for the presence of a microbe. Alternatively, the swab may be used to culture a liquid or plate (e.g., agar) medium in order to promote the growth of any pathogen for later testing.

In general, samples would be maintained in conditions similar to those existing at the source of the sample. Thus, samples would be maintained in culture conditions that mimic the conditions at the source of the sample.

In general, the in situ hybridization methods used herein include the steps of fixing a biological or non-biological sample, hybridizing labeled oligonucleotides to target RNA contained within the fixed sample, washing to remove non-specific binding. In situ hybridization assays and methods for sample preparation are well known to those of skill in the art and need not be described in detail here. Such methods can be found in, for example, Amann R. et al., 1995, Microbiol. Rev. 59(1): 143-69; Bruns and Berthe-Corti, 1998, Microbiology 144, 2783-2790; Vesey G. et al., 1998, J. App. Microbiol. 85, 429-440; and Wallner G. et al., 1995, Appl. Environ. Microbiol. 61(5): 1859-1866.

Fluorescence in situ hybridization (FISH) offers many advantages over radioactive and chromogenic methods for detecting hybridization. Not only are fluorescence techniques fast and precise, they allow for simultaneous analysis of multiple signals that may be spatially overlapping. Through use of appropriate optical filters, it is possible to distinguish multiple different fluorescent signals in a single sample using their excitation and emission properties alone. Methods for combinatorial labeling are described in, e.g., see, Ried et al., 1992, Proc. Natl. Acad. Sci. USA 89, 1388-1392; Tanke, H. J. et al, 1999, Eur. J. Hum. Genet. 7: 2-11. By using combined binary ratio labeling (COBRA) in conjunction with highly discriminating optical filters and appropriate software, over 40 signals can be distinguished in the same sample, see, e.g., Wiegant J. et al., 2000, Genome Research, 10 (6), 861-865.

In certain embodiments, microbial cells are harvested from a biological or non-biological sample using standard techniques, some of which are described in the previous section. For example, cells can be harvested by centrifuging a sample and resuspending the pelleted cells in, for example, phosphate-buffered saline (PBS). After re-centrifuging the cell suspension to obtain a cell pellet, the cells can be fixed in a solution such as an acid alcohol solution, an acid acetone solution, or an aldehyde such as formaldehyde, paraformaldehyde, or glutaraldehyde. For example, a fixative containing methanol and glacial acetic acid in a 3:1 ratio, respectively, can be used as a fixative. A neutral buffered formalin solution also can be used (e.g., a solution containing approximately 1% to 10% of 37-40% formaldehyde in an aqueous solution of sodium phosphate). Slides containing the cells can be prepared by removing a majority of the fixative, leaving the concentrated cells suspended in only a portion of the solution. Methods for fixing microbes are known in the art and can be adapted to suit different types of microbes, if needed. Determination of suitable fixation/permeabilization protocols are carried out routinely in the art.

In some embodiments, a secondary detection method may be employed to amplify the signal, for example, by using a series of multiply labeled oligonucleotides that recognize adjacent sequences. However, oligonucleotide probes can be sufficiently sensitive to detect a single RNA transcript in situ. In addition, molecular beacons that are labeled with a fluorophore and a quencher can provide the sensitivity required to detect about 10 molecules of RNA in a single cell in situ without the need for amplification.

When more than one label is used, fluorescent moieties that emit different signal can be chosen such that each label can be distinctly visualized and quantitated. For example, a combination of the following fluorophores may be used: 7-amino-4-methylcoumarin-3-acetic acid (AMCA), Texas Red™ (Molecular Probes, Inc.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a, 4a diaza-3-indacenepropionic acid, eosin-5-isothiocyanate, erythrosin-5-isothiocyanate, and Cascade™ blue acetylazide (Molecular Probes, Inc.). Hybridized oligonucleotides can be viewed with a fluorescence microscope and an appropriate filter for each fluorophore, or by using dual or triple band-pass filter sets to observe multiple fluorophores. See, for example, U.S. Pat. No. 5,776,688.

Hybridized oligonucleotides also can be labeled with biotin, digoxygenin, or radioactive isotopes such as ³²P and ³H, although secondary detection molecules or further processing may then be required to visualize the hybridized oligonucleotides and quantify the amount of hybridization. For example, an oligonucleotide labeled with biotin can be detected and quantitated using avidin conjugated to a detectable enzymatic marker such as alkaline phosphatase or horseradish peroxidase. Enzymatic markers can be detected and quantitated in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Prior to in situ hybridization, the oligonucleotides may be denatured. Denaturation is typically performed by incubating in the presence of high pH, heat (e.g., temperatures from about 70° C. to about 95° C.), organic solvents such as formamide and tetraalkylammonium halides, or combinations thereof.

Permeabilized/fixed cells are contacted with labeled oligonucleotides under in situ hybridizing conditions. “In situ hybridizing conditions” are conditions that facilitate annealing between a nucleic aid and the complementary nucleic acid. Hybridization conditions vary, depending on the concentrations, base compositions, complexities, and lengths of the probes, as well as salt concentrations, temperatures, and length of incubation. For example, in situ hybridizations typically are performed in hybridization buffer containing 1-2×SSC, 50% formamide, and blocking DNA to suppress non-specific hybridization. In general, hybridization conditions include temperatures of about 25° C. to about 55° C., and incubation times of about 0.5 hours to about 96 hours. Suitable hybridization conditions for a set of oligonucleotides and target microbe can be determined via experimentation which is routine for one of skill in the art.

The microbes might be present in a suspension or alternatively, the microbes may be immobilized on a substrate. A suspension or a solution containing the microbes might be preferred where an automated or a semi-automated system is used for sorting the microbes into different types of microbes, for example, by fluorescence-activated cell sorter. Alternatively, immobilization of the microbes might be desirable in applications where additional microscopic features, such as, morphology of the microbe is to be assessed. Obviously, a suspension of microbes might be sorted into different types of microbes based on the predetermined optically detectable signature, followed by immobilization of the microbes.

The contacted sample can be read using a variety of different techniques, e.g., by microscopy, flow cytometry, fluorimetry etc.

Microscopy, such as, light microscopy, fluorescent microscopy or confocal microscopy, is an established analytical tool for detecting light signal(s) from a sample. In embodiments in which oligonucleotides are labeled with a fluorescent moiety, reading of the contacted sample to detect hybridization of labeled oligonucleotides may be carried out by fluorescence microscopy. Fluorescent microscopy or confocal microscopy used in conjunction with fluorescent microscopy has an added advantage of distinguishing multiple labels even when the labels overlap spatially.

Flow cytometers are well known analytical tools that enable the characterization of particles on the basis of light scatter and particle fluorescence. In a flow cytometer, particles are individually analyzed by exposing each particle to an excitation light, typically one or more lasers, and the light scattering and fluorescence properties of the particles are measured. Particles, such as molecules, analyte-bound beads, individual cells, or subcomponents thereof, typically are labeled with one or more spectrally distinct fluorescent dyes, and detection is carried out using a multiplicity of photodetectors, one for each distinct dye to be detected. The detection creates a readable output, e.g. type of signal or fluorescent intensity, etc. Flow cytometers are commercially available from, for example, BD Biosciences (San Jose, Calif.). Methods of reading fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Col, Inc. (1978), pp. 296-361. A variety of FACS systems are known in the art and can be used in the methods of the invention (see e.g., WO99/54494, filed Apr. 16, 1999; U.S. Ser. No. 20010006787, filed Jul. 5, 2001, each are incorporated herein by reference).

In embodiments in which oligonucleotides are labeled with a fluorescent moiety, reading of the contacted sample to detect hybridization of labeled oligonucleotides may be carried out by fluorescence activated cell sorter (FACS). In addition to detecting hybridization and sorting cells based on their optically detectable signature, FACS may optionally provide an enumeration of microbes of a particular type present in a sample. This would facilitate estimate of titer of the microbe in a sample. Alternatively or in addition, the titer of the microbe in a sample might be estimated by reading the sample in a spectrophotometer.

In certain embodiments, the cells are sorted based on the magnitude or intensity of the fluorescent signal. For example, cells may be sorted into different fractions based on the magnitude or intensity of signal(s) produced from the cells. Cell can be sorted into different fractions where the intensity of a first signal in the different fractions increases by, for example, two-folds. These fractions can then be sorted based on the intensity of a second signal, where the intensity of the second signal in the different fractions also increases by two-folds. In this manner, cells with predetermined optically detectable signatures of ratio of the magnitude of the first signal to that of the second signal would be sorted into different fractions and identified on the basis of the predetermined optically detectable signature.

In certain embodiments, the specific type of labels used for each sample is selected such that the detection characteristic of each label does not interfere with the detection characteristic of any other label (whether the labels are present in the same cell or are present in different cells in the same sample) upon flow cytometric analysis. Such parameters are routinely considered by those of skill in the art of multi-parameter flow cytometry. Parameters that may influence the choice of label to employ include, but are not limited to, overlap of the labels present in the multiplex sample (e.g., fluorescence emission overlap of distinct fluorescent labels), overlap of the detection characteristics of one label with that of another label, excitation wavelength, fluorescence intensity, and the detector channels available in the flow cytometer being used for analysis. The appropriate corresponding detection channels may be selected based on the predetermined optically detectable signatures that are to be detected. Methods for selection of appropriate detection channels are well known and are within the ability of one of skill in the art of flow cytometry.

In certain embodiments, the label is a fluorescent dye. Fluorescent dyes (fluorophores) suitable for use as labels in the present method can be selected from any of the many dyes suitable for use in imaging applications, especially flow cytometry. A large number of dyes are commercially available from a variety of sources, such as, for example, Molecular Probes (Eugene, Oreg.) and Exciton (Dayton, Ohio), that provide great flexibility in selecting a set of dyes having the desired spectral properties. Examples of fluorophores include, but are not limited to, 4-acetamido-4′-isothiocyanatostilbene-2,2′ disulfonic acid; acridine and derivatives such as acridine, acridine orange, acridine yellow, acridine red, and acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-amino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy5, Cy5.5, and Cy7; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; Alexa-Fluor dyes (e.g., Alexa Fluor 350, Alexa Fluor 430, Alexa Fluor 488, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, Alexa Fluor 750), Pacific Blue, Pacific Orange, Cascade Blue, Cascade Yellow; Quantum Dot dyes (Quantum Dot Corporation); Dylight dyes from Pierce (Rockford, Ill.), including Dylight 800, Dylight 680, Dylight 649, Dylight 633, Dylight 549, Dylight 488, Dylight 405; or combinations thereof. Other fluorophores or combinations thereof known to those skilled in the art may also be used, for example those available from Molecular Probes (Eugene, Oreg.) and Exciton (Dayton, Ohio).

Fluorescence in a sample can be measured using a fluorimeter. In general, excitation radiation, from an excitation source having a first wavelength, passes through excitation optics. The excitation optics causes the excitation radiation to excite the sample. In response, fluorescent molecules in the sample emit radiation that has a wavelength that is different from the excitation wavelength. Collection optics then collects the emission from the sample. The device can include a temperature controller to maintain the sample at a specific temperature while it is being scanned. A multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Table 1 below provides exemplary combinations of fluorophores that may be used together in combinations of 2, 3 or 4. This table is by no means comprehensive. In Table 1, 20 different 2 dye combinations, 9 different 3 dye combinations, and 8 different 4 dye combinations are denoted (read vertically; filled-in black box indicates dyes in the combination).

TABLE 1
Exemplary Dye Combinations (AF = Alexa Fluor).

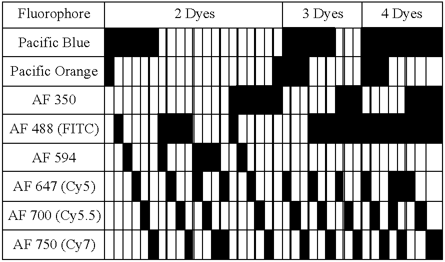

Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference).

The identity of a microbe is determined on the basis of the predetermined optically detectable signature associated with the microbe. This determination may be carried out either manually or in an automated system. Because the optically detectable signature associated with known microbes are known, the predetermined optically detectable signature associated with a microbe in a sample can be matched to the signature associated with known microbes. The matching may be performed by using computer-based analysis software known in the art. Determination of identity may be done manually (e.g., by viewing the data and comparing the signatures by hand), automatically (e.g., by employing data analysis software configured specifically to match optically detectable signature), or a combination thereof. In certain embodiments, the detection of an optically detectable signature identifies the microbe. Such an embodiment might be automated to generate a “yes” answer for the sample analysis if an optically detectable signature is associated with a microbe and a “no” answer if an optically detectable signature is not associated with a microbe.

A list, look-up table or a database listing the different sets of labeled oligonucleotides and the predetermined optically detectable signals they provide to microbes of known identity may be provided by pre-testing the different sets of labeled oligonucleotides with the known microbes. In certain embodiments, in silico determination of optically detectable signature may be used to populate the list or look-up table. This list, look-up table or a database may be searchable manually or automatically.

### Compositions

Provided herein are compositions comprising a set of at least two labeled oligonucleotides as described above. These labeled oligonucleotides: i) hybridize to different RNA molecules of a microbe at sites that are unique to the microbe; ii) provide a predetermined optically detectable signature that identifies the microbe when the labeled oligonucleotides are hybridized to the different RNA molecules of the microbe, and iii) do not hybridize to ribosomal RNA of the microbe. Also provided herein are compositions comprising a plurality of sets of oligonucleotides, where the oligonucleotides of said sets, when labeled, comprise a plurality of sets of labeled oligonucleotides, where each set of the plurality of sets of labeled oligonucleotides hybridizes to RNA molecules of different microbes and provides a predetermined optically detectable signature that identifies the different microbes, where the labeled oligonucleotides in each of the plurality of sets: i) hybridize to different RNA molecules of the different microbes at sites that are unique to the different microbes; ii) provide a predetermined optically detectable signature that identifies the different microbes when the labeled oligonucleotides are hybridized to the different RNA molecules of the different microbes, and iii) do not hybridize to ribosomal RNA of the different microbes. In certain embodiments, the oligonucleotides of a plurality of sets of oligonucleotides are present in a solution. In certain embodiments, the oligonucleotides of the plurality of sets of oligonucleotides are present on an array, where a single type of set is present on a single array. In embodiments where the oligonucleotides are present on an array, the oligonucleotides are attached to the array by a cleavable linker that is cleaved to release the oligonucleotides. In certain other embodiments, the oligonucleotides are provided in a labeled form. FIG. 3 depicts an embodiment of the subject method and compositions. Sets of labeled oligonucleotides are provided attached to different arrays 10. Each set provides a different predetermined optically detectable signature to a microbe of a particular type. Thus set 1 provides a signature of 1:1 which is the ratio of magnitude of a first signal to that of a second signal, and so on. The labeled oligonucleotides are derived into solution phase in different containers. At this step, the labeling of the oligonucleotides may optionally be checked by reading the signals from the labeled oligonucleotides by, for example, using a fluorimeter. These sets are mixed together before contacting a sample under in situ hybridization conditions.

### Kits

Also contemplated are kits for practicing the above described subject method. The subject kits contain at least a subject oligonucleotides composition. The oligonucleotides may be supplied in a solution or may be present in an array. The oligonucleotides may be supplied in unlabeled or labeled forms. The kit may also contain reagents for labeling oligonucleotides, amplifying oligonucleotides, reagents for permeabilizing microbes, in situ hybridization reagents, microbes that serve as positive controls for the oligonucleotides supplied in the kit, etc. The various components of the kit may be present in separate containers or certain compatible components may be pre-combined into a single container, as desired.

In addition to above-mentioned components, the subject kits may further include instructions for using the components of the kit to practice the subject methods, i.e., instructions for sample analysis. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. In certain embodiments, the instructions for sample analysis may include the look-up table or include a web address where the look-up table can be viewed or downloaded. As with the instructions, this means for obtaining the instructions is recorded on a suitable substrate.

### Utility

The above described method is useful for the analysis of samples in a variety of diagnostic, drug discovery, and research applications. The above described method is useful for the analysis of biological samples. The term “biological sample”, as used herein, refers to a sample obtained from an organism or from components (e.g., cells) of an organism. The sample may be of any biological tissue or fluid. In some cases, the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells there from. Biological samples may also include sections of tissues such as frozen sections taken for histological purposes. The subject method also finds use in determining the identity of microbes in water, sewage, air samples, food products, including animals, vegetables, seeds etc., soil samples, plant samples, microbial culture samples, cell culture samples, tissue culture samples, as well as in human medicine, veterinary medicine, agriculture, food science, bioterrorism, and industrial microbiology etc. The subject method allows identification of hard to culture microbes since culturing the microbes is not necessary. Consequently, the subject method provides for a rapid detection of microbes in a sample with no waiting period for culturing microbes.

Microbes that might be identified using the subject methods, compositions and kits include but are not limited to: a plurality of species of Gram (+) bacteria, plurality of species of Gram (−) bacteria, a plurality of species of bacteria in the family Enterobacteriaceae, a plurality of species of bacteria in the genus Enterococcus, a plurality of species of bacteria in the genus Staphylococcus, and a plurality of species of bacteria in the genus Campylobacter, Escherichia coli (E. coli), E. coli of various strains such as, K12-MG1655, CFT073, O157:H7 EDL933, O157:H7 VT2-Sakai, etc., Streptococcus pneumoniae, Pseudomonas aeruginosa, Staphylococcus aureus, coagulase-negative staphylococci, a plurality of Candida species including C. albicans, C. tropicalis, C. dubliniensis, C. viswanathii, C. parapsilosis, Klebsiella pneumoniae, a plurality of Mycobacterium species such as M. tuberculosis, M. bovis, M. bovis BCG, M. scrofulaceum, M. kansasii, M. chelonae, M. gordonae, M. ulcerans, M. genavense, M. xenoi, M. simiae, M. fortuitum, M. malmoense, M. celatum, M. haemophilum and M. africanum, Listeria species, Chlamydia species, Mycoplasma species, Salmonella species, Brucella species, Yersinia species, etc. Thus, the subject method enables identification of microbes to the level of the genus, species, sub-species, strain or variant of the microbe.

The subject methods, compositions and kits are also useful in identifying multiple different microbes in a single sample, simultaneously. This multiplexing aspect of the subject method offers the advantages of conserving time, reagents and sample size.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of sample analysis, comprising:
a) contacting a sample comprising a microbe with a set of at least two labeled oligonucleotides under in situ hybridization conditions to produce a contacted sample, wherein said labeled oligonucleotides:
i) hybridize to different RNA molecules of said microbe at sites that are unique to said microbe;
ii) provide a predetermined optically detectable signature that identifies said microbe when said labeled oligonucleotides are hybridized to said different RNA molecules of said microbe, and
iii) do not hybridize to ribosomal RNA of said microbe;
b) reading said contacted sample to detect hybridization of said labeled oligonucleotides; and
c) determining the identity of said microbe on the basis of said predetermined optically detectable signature, wherein said predetermined optically detectable signature indicates the identity of said microbe in said sample.

2. The method of claim 1, wherein all of said labeled oligonucleotides in said set comprise the same label, wherein said label provides said optically detectable signature.

3. The method of claim 1, wherein said set of labeled oligonucleotides comprises a first population of labeled oligonucleotides and a second population of labeled oligonucleotides, wherein said first population is labeled with a first label that produces a first signal and said second population is labeled with a second label that produces a second signal is that distinguishable from said first signal.

4. The method of claim 3, wherein the ratio of the magnitude of said first and second signals, when said first and second populations of labeled oligonucleotides are hybridized to said different RNA molecules, provides said predetermined optically detectable signature that identifies said microbe.

5. The method of claim 1, wherein said labeled oligonucleotides are labeled with a fluorescent moiety.

6. The method of claim 1, wherein said reading is carried out by using a fluorescence microscope.

7. The method of claim 1, wherein said determining is carried out by matching said predetermined optically detectable signature associated with said microbe to optically detectable signal associated with known microbes.

8. The method of claim 1, wherein said labeled oligonucleotides hybridize to different RNA molecules at sites that are unique to the genus of said microbe.

9. The method of claim 1, wherein said labeled oligonucleotides hybridize to different RNA molecules at sites that are unique to the species of said microbe.

10. The method of claim 1, wherein said labeled oligonucleotides hybridize to different RNA molecules at sites that are unique to the strain of said microbe.

11. The method of claim 1, comprising:
a) contacting said sample with a plurality of sets of labeled oligonucleotides under in situ hybridization conditions to produce a contacted sample, wherein each set of said plurality of sets hybridizes to RNA molecules of different microbes and provides a predetermined optically detectable signature that identifies said different microbes, wherein said labeled oligonucleotides in each of said plurality of sets:
i) hybridize to different RNA molecules of said different microbes at sites that are unique to said different microbes;
ii) provide a predetermined optically detectable signature that identifies said different microbes when said labeled oligonucleotides are hybridized to said different RNA molecules of said different microbes, and
iii) do not hybridize to ribosomal RNA of said different microbes;
b) reading said contacted sample to detect hybridization of said labeled oligonucleotides; and
c) determining the identity of said microbe on the basis of said predetermined optically detectable signal, wherein said predetermined optically detectable signature indicates the identity of said microbe in said sample.

12. The method of claim 11, wherein said sample comprises different microbes and wherein step c) comprises determining the identity of said different microbes on the basis of said predetermined optically detectable signature, wherein said predetermined optically detectable signature indicates the identity of said different microbes in said sample comprising different microbes.

13. The method of claim 11, wherein each of said plurality of sets of labeled oligonucleotides comprises a first population of labeled oligonucleotides and a second population of labeled oligonucleotides, wherein said first population is labeled with a first label that produces a first signal and said second population is labeled with a second label that produces a second signal, wherein said first signal is distinguishable from said second signal.

14. The method of claim 13, wherein the ratio of said first and second signals provides said predetermined optically detectable signature that identifies said different microbes, wherein said predetermined optically detectable signature is different for a different microbe.

15. The method of claim 11, wherein all of said labeled oligonucleotides in a set are labeled with the same label and said same label is distinguishable from said same label of another set of labeled oligonucleotides.

16. A composition of oligonucleotides, comprising a plurality of sets of oligonucleotides, wherein each set of said plurality of sets of oligonucleotides hybridizes to RNA molecules of different microbes, wherein said oligonucleotides in each of said plurality of sets:
i) hybridize to different RNA molecules of said different microbes at sites that are unique to said different microbes; and
ii) do not hybridize to ribosomal RNA of said different microbes.

17. The composition of claim 16, wherein said oligonucleotides of a plurality of sets of oligonucleotides are present in a solution.

18. The composition of claim 16, wherein said oligonucleotides of said plurality of sets of oligonucleotides are present on a plurality of arrays.

19. A kit for sample analysis, comprising:
a) a plurality of sets of labeled oligonucleotides, wherein said each set of said plurality of sets of labeled oligonucleotides hybridizes to RNA molecules of different microbes and provides a predetermined optically detectable signature that identifies said different microbes, wherein said labeled oligonucleotides in each of said plurality of sets:
i) hybridize to different RNA molecules of said different microbes at sites that are unique to said different microbes;
ii) provide a predetermined optically detectable signature that identifies said different microbes when said labeled oligonucleotides are hybridized to said different RNA molecules of said different microbes, and
iii) do not hybridize to ribosomal RNA of said different microbes; and
b) reagents for performing in situ hybridization.

20. The kit of claim 19, further comprising a set of labeled oligonucleotides that hybridize to different RNA molecules of a known microbe at sites unique to said known microbe and said known microbe.
